(19) United States
(12) Patent Application Publication
(10) Pub. No.: US 2010/0291695 A1
(43) Pub. Date: Nov. 18, 2010

(54) DETECTION OF NUCLEIC ACID SEQUENCE MODIFICATION

(76) Inventor: David Isaacs, West Hampstead (GB)

Correspondence Address: LANDO & ANASTASI, LLP, ONE MAIN STREET, SUITE 1100, CAMBRIDGE, MA, 02142, US

(21) Appl. No.: 12/095,696

(22) PCT Filed: Nov. 30, 2006
(86) PCT No.: PCT/GB06/50421

§ 371 Date: Aug. 9, 2010

(30) Foreign Application Priority Data
Nov. 30, 2005 (GB) 0524299.5

(51) Int. Cl. G01N 33/00

(52) U.S. Cl. 436/94; 422/68.1

(57) ABSTRACT

The invention relates to a non-PCR based method for the detection of a nucleotide modification in a nucleic acid sample comprising contacting a solution comprising nucleic acid sample with a nucleic acid probe in a temperature-controlled and UV illuminated container and measuring the UV absorption of the nucleic acid sample/nucleic acid probe complex.

The invention relates to a method for nucleic acid detection, in particular to non-PCR based detection of a nucleotide modification in a nucleic acid sequence.

### BACKGROUND

The detection of nucleic acid sequence modifications, has many applications and is a particularly important tool in the diagnosis of disease. Most techniques employ the Polymerase Chain Reaction (PCR), which enables the amplification of very small amounts of complex genetic material (U.S. Pat. No. 4,683,202). PCR includes the steps of denaturation, annealing and extension and is a well-known tool in the field of molecular biology. Traditional PCR methods analyse the product by agarose gel electrophoresis. The disadvantage is that this method is time consuming and shows low sensitivity. The basic PCR method has been developed further and methods are now available for detecting sequence-specific PCR products in real time. One such method is the TaqMan® assay wherein detection of PCR products is based on the detection of fluorescence of a reporter. However, despite its advantages, the TaqMan® assay also has some disadvantages. For example, sequence data for the construction of probes must be available. Therefore, the costs for the assay are particularly high when different probes need to be synthesised for the detection of different sequences. Another method for real-time PCR commonly used employs a dye (SYBR Green®), which binds specifically to double-stranded DNA, but not to single-stranded DNA. However, this method has the disadvantage that the dye is non-specific and can generate false positive signals. Other methods use molecular beacons or scorpions but similar to the TaqMan® assay, these methods are complex and expensive.

Nucleic acid modifications include short tandem repeats (STR) and single nucleotide polymorphisms (SNPs). SNPs are DNA sequence variations that occur when a single nucleotide (A, T, C or G) in the genome sequence is altered. For a variation to be considered a SNP, it must occur in at least 1% of the population. Allele frequencies vary greatly, also amongst different populations. SNPs, which make up about 90% of all human genetic variation, occur every 100 to 300 bases along the 3-billion-base human genome. Because SNPs are usually only present in two forms, the allele that is more rare is referred to as mutant or minor allele and the most common allele is referred to as wild type allele. SNPs are primarily bi-allelic (i.e. there are two possible alleles at one locus) but may also be tri-allelic (i.e. two independent mutation events have occurred at the same time). Two of every three SNPs involve the replacement of cytosine (C) with thymine (T). SNPs can occur in both coding (gene) and non-coding regions of the genome (extronic or intronic).

Although more than 99% of human DNA sequences are the same across the population, variations in DNA sequence can have a major impact on how humans respond to disease, pathogens and therapies. This makes SNPs of great value for biomedical research and for developing pharmaceutical products or medical diagnostics. Therefore, the provision of an efficient, precise, cheap and user-friendly method for the detection of SNPs can be of great value. Current methods used to analyse SNPs include PCR followed by sequencing, microarrays and mass spectrometry. However, in particular microarrays and mass spectrometry are complex and expensive. Therefore, there is a need for an alternative and improved method for analysing SNPs.

Most DNA molecules show a relative increase of 1.4 in absorbance at 260 nm upon denaturation, which is known as the hyperchromic effect. The hyperchromic effect can be explained by the specific stacking of the nitrogenous bases in the double helix. When the bases are stacked on top of one another in the double helix, they interact relatively poorly with light. Increased thermal vibrations at high temperatures destabilize the DNA double helix and ultimately cause the two DNA strands to separate. When the two strands separate, the pentose sugars are free to rotate about their phophodiester linkages, thereby resulting in less effective stacking interactions between the nitrogenous bases, and increased interaction with light. The absorption changes are therefore those that result from the transition of an ordered double-helix DNA structure to a denatured state or random, unpaired DNA strands. The process of DNA dissociation can, therefore, be characterized by monitoring the UV-absorbing properties of DNA under various conditions.

The dissociation of double stranded DNA (dsDNA) helix structure into its single stranded form (ssDNA) is called melting and it occurs at a temperature that is a function of the base composition of the DNA. The melting temperature (Tim value) is defined as that temperature at which half of the helical structure is lost. The melting temperature strongly depends on the base composition in the studied DNA as well as chemical criteria such as pH and ionic conditions. A large number of G-C base pairs increases the Tm of DNA, while DNA with mainly A-T composition has a lower melting point. The nature of base pair interaction can serve as an explanation for that phenomenon. G-C base pairs have three connecting hydrogen bonds compared to A-T base pairs which have two hydrogen bonds only. Thus, breaking down of stronger G-C interactions requires more energy. Measurement of DNA absorption upon changing of temperature allows the precise determination of the dsDNA's stability.

The invention relates to a method for nucleic acid detection, in particular to non-PCR based detection of a nucleic acid modification. The method aims to improve current methods which make use of PCR as PCR-based methods have a number of disadvantages, for example the error rate introduced by many thermostable polymerases.

WO 03/036302 discloses a method for monitoring the folding and unfolding of proteins and an apparatus for analysing temperature-dependent configurations of proteins. The contents of WO 03/036302 are hereby incorporated by reference.

### SUMMARY OF THE INVENTION

In a first aspect, the invention relates to a method for the detection of a nucleotide modification in a nucleic acid sample comprising:
a) contacting a solution comprising a nucleic acid sample with a nucleic acid probe in a temperature-controlled and UV illuminated container;
b) measuring the UV absorption of the nucleic acid sample/nucleic acid probe complex;
   wherein the method does not comprise nucleic acid amplification.

Viewed from a second aspect, the invention relates to the use of the method according to the invention for diagnosing a patient as having a disease or being susceptible to it.

In accordance with a further aspect, the invention relates to the use of an apparatus in the method of the invention having a plurality of temperature-controlled channels.

### DESCRIPTION OF THE INVENTION

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In accordance with a first aspect of the invention, there is provided a method for the detection of a nucleotide modification in a nucleic acid sample comprising:
a) contacting a solution comprising a nucleic acid sample with a nucleic acid probe in a temperature-controlled and UV illuminated container;
b) measuring the UV absorption of the nucleic acid sample/nucleic acid probe complex;
   wherein the method does not comprise amplification of the nucleic acid.

Thus, the method of the invention does not comprise PCR amplification.

The first step of the method thus comprises contacting a solution comprising a nucleic acid sample with a nucleic acid probe. Preferably, the probe is designed so that it is complementary to a target sequence in the nucleic acid sample and therefore, it will form a complex with the nucleic acid sample. The binding of a probe to a target is illustrated in FIG. 4.

Preferably, the method comprises measuring the Tm of the nucleic acid sample/nucleic acid probe complex.

The key issue of the invention is to use the hyperchromic effect, the fact that single stranded DNA absorbs more energy than double stranded DNA. The invention comprises measuring the temperature at which a complementary probe, designed to probe a particular location on the primary structure, dissociates or melts from the target sequence in the nucleic acid sample. The probe is, for example, designed to bind to the possible site of a point mutation, such a single nucleotide polymorphism (SNPs). Should the mutation be present in the target sequence and the primer probe complementary to the wild type sequence, the probe will bind with a lower energy than should the sequences be fully complementary. Alternatively, should the probe contain a base complementary to the SNP sequence, it will bind to the wildtype sequence with less energy than to the SNP sequence. In these cases the melting temperature will be lower than in any case where the duplex formed between the target sequence in the sample and the probe is fully complementary. This difference in melting temperature will be exploited to validate the presence or absence of a point mutation. The invention provides label free imaging although in certain embodiments of the invention, the probe may be labelled.

Thus, the invention makes use of the hyperchromic effect of nucleic acid. According to the invention, the solution is illuminated with UV light to determine the amount of light absorbed by the nucleic acid sample at a given temperature. Thus, the amount of light absorbed by the nucleic acid sample at one or more temperatures or at a range of temperatures applied is determined. The amount of light absorbed indicates the presence of double stranded nucleic acid and thus the presence or absence of a nucleotide modification.

The term container refers to a holding for the nucleic acid sample, such as a chamber, capillary or channel for carrying out the reaction which can be temperature-controlled and UV illuminated according to the invention. For example, the mixture comprising the nucleic acid sample and probe may be held in a micro-centrifuge tube which is placed in a thermocycler. In another embodiment, the mixture is passed along a channel which may form part of a microfluidic chip. The container may be made from a variety of UV transparent materials, such as, but not limited to, plastic, quartz or glass.

In one embodiment, the container used in the method of the invention is a temperature-controlled and UV illuminated channel. Thus, the solution comprising the nucleic acid sample/nucleic acid probe complex may be passed along the channel and different temperatures may be applied along the length of the channel. In a preferred embodiment, the solution comprising the nucleic acid sample/nucleic acid probe complex is imaged along the channel.

The term temperature-controlled according to the invention refers to controlling the temperature in the container. For example, a temperature profile is applied to the length of the channel and thus to the solution within the channel. According to the invention, the temperature is controlled so that a range of temperatures can be applied to the solution along the length of the channel. Thus, a specified temperature can be applied to the channel (and thus the solution) at any given point along the length of a channel. Thus, it is possible to apply a specific temperature to the solution at each possible position along the length of the channel. For example, the channel typically spans 36 mm between the temperature controlling Peltier cells. Temperature resolution can be adjusted to suit experimental conditions, but resolution of 0.1° C. per millimetre or lower may be used.

The method of the invention allows fast reaction times, because the small-volume fluid elements can be heated or cooled to the required temperature within 100 milliseconds. Moreover, operation within a serial or parallel format allows multiple reactions to be performed simultaneously.

According to the invention, a temperature gradient may be applied to the channel so that the temperature at the start point is higher than at the end point. Alternatively, the gradient may be defined so that the temperature is at its highest at the start point and increases along the length of the channel to reach a minimum at the end of the channel. Alternatively, a range of temperatures is applied so that the channel is controlled by a multi-value gradient. The temperature may also be raised across the whole channel, so that the entire sample is evenly heated. Accordingly, in one embodiment, the temperature applied is a temperature gradient. In another embodiment, the temperature applied is a multi value gradient. In another embodiment, the whole channel is evenly heated.

Preferably, the temperature applied is in the range of from about 20° C. to about 110° C.

The channel may be temperature-controlled by the use of heating elements, such as thermal heating strips or Peltier cells. The temperature may be monitored with temperature detectors located along the length of the channel. WO 03/036302 discloses an apparatus for analysing temperature-dependent configurations of proteins and complexes of proteins with biological factors. In certain embodiments of the method of the invention, the method may be carried out using the apparatus similar to that described in WO 03/036302. Accordingly, in a further aspect, the invention also relates to the use of an apparatus for the detection of nucleic acid having a plurality of channels. In a preferred embodiment, the apparatus comprises a multi-lane chip having a plurality of channels extending along a length thereof, each channel being arranged for the detection of the nucleic acid sequence to be analysed and comprising heating elements associated with the chip for creating a temperature profile along the channel or channels.

The width of the channel according to the invention is in the range of 10s of micrometers to hundreds of micrometers. Microfluidic systems typically range from 50×50 μM to 400×400 μMs, with a range of aspect ratios around these values. In a preferred embodiment, the channel is 50 micrometers wide and 200 micrometers deep. The channel may arranged in such a way that the solution moves due to mechanisms such as a pressure pumping system or an electrokinetic injection to enable movement of the solution along the channel although other mechanisms are also within the scope of the invention.

Preferably, the channel is parallel sided and illuminated with UV light from above. Preferably, the channel forms part of a chip and comprises underlying optical detectors. The detector may be connected to a computer system so that the output of the detector can be analysed. Thus, UV light passes through the solution in the channel and through the chip and is detected by the underlying optical detector. Absorption is measured at 260 nm to detect the presence of double stranded or single stranded nucleic acid. Thus, absorption is measured as a function of temperature.

The ability to precisely control the temperature at different points, either along the channel or in a capillary, is an important feature of the method of the invention as it enables the control of the temperature at any given position of the nucleic acid sample, for example along the length of the channel. Accordingly, depending on the position of the nucleic acid in the channel, the temperature can be monitored.

According to the invention, UV absorption is measured as a function of temperature to detect the presence or absence of single-stranded or double-stranded nucleic acid. Furthermore, as the hyperchromic effect depends on the nucleic acid sequence, the methods of the invention can also be used to analyse the sequence of the nucleic acid sample.

According to the invention, the term nucleic acid sample refers to a sample comprising DNA or RNA. The nucleic acid sample may be comprised in a solution which may, for example, contain buffers. The DNA may comprise cDNA and RNA may comprise mRNA or siRNA. Preferably, the nucleic acid sample comprises single-stranded DNA or RNA or double-stranded DNA or RNA. If the nucleic acid sample comprises double stranded nucleic acid, then the nucleic acid may be firstly denatured by applying a temperature of about 94° C. to the solution in the container. In one embodiment, the nucleic acid comprises native secondary structural elements or is in its denatured form. In one embodiment, the nucleic acid sample may comprise nucleic acid isolated from a microorganism, animal or plant. In another embodiment, the nucleic acid is a synthetic sequence, for example a part of a vector or oligonucleotide. In a further embodiment, the nucleic acid sample comprises animal or plant cells or cells of a microorganism.

According to the methods of the invention, detection may be in real time.

A nucleotide modification according to the invention designates a modification in the sequence of the nucleic acid, for example, it may be the substitution, deletion or addition of a nucleotide or base pair, for example due to a mutation. According to the invention, one or more nucleotide modifications may be detected. In particular, the modification may be STR, SNP, or a Targeted Genetic Modification (GM) step. Preferably, the nucleotide modification is a SNP. SNPs are single base pair positions in genomic DNA at which different sequence alternatives (alleles) exist in normal individuals in some population(s), wherein the least frequent allele has an abundance of 1% or greater. As SNPs are often associated with disease, the detection of SNPs has particular value in the field of diagnostics. Accordingly, in one embodiment of the invention, the SNP is associated with disease. The detection of SNPs according to the invention may thus be carried out by using a method comprising passing a solution comprising a nucleic acid sample and a nucleic acid probe along a temperature-controlled and UV illuminated channel and measuring UV absorption of the nucleic acid sample/probe complex. Thus, the presence or absence of single-stranded or double-stranded nucleic acid is determined. If a SNP is present, the sample/probe complex will be less stable and exhibit a lower melting temperature. Even if the difference is very small, the temperature resolution of the method of the invention will allow the detection of single stranded or double stranded DNA and therefore the determination of the presence of the SNP.

A nucleic acid probe according to the invention is defined as a nucleic acid fragment which specifically anneals to the sequence of interest. Therefore, the sequence of the target is preferably known. The sequence of the nucleic acid probe is thus designed so that it is complementary to the sequence of interest. For example, the nucleic acid probe may be a synthetic oligonucleotide or a cDNA fragment which anneals to a gene sequence of interest. In another embodiment, the nucleic acid probe may comprise RNA. Nucleic acid probes used according to the invention generally comprise 10 to 30, preferably 15 to 25 nucleotides, but may be larger for secondary structural or other applications. According to the method of the invention, the nucleic acid probe may also be labelled to provide a further level of detection. Such labels are known to the skilled person and include fluorescent dyes or radioactive labels. The precise temperature control of the channel enables to accurately adjust of the temperature required for the specific probe used.

SNPs have been suggested to be involved in disease, therefore they can help to predict susceptibility to a particular condition, such as Alzheimer's, heart disease, diabetes or cancer. Association studies are used to link SNPs to such conditions. The method of the invention provides a way of testing for the presence or absence of a SNP in a candidate allele therefore giving an indication of disease susceptibility of the patient. Accordingly, in another aspect, the invention relates to the use of the method described herein for diagnosing a patient as having a disease or being susceptible to it. SNPs may also be used to develop a personalised drug treatment regime taking into account the individual profile of a patient. Accordingly, the method of the invention may be used in developing a particular drug treatment regime. The development of a robust and cheap non-PCR based SNP validation will be of immense value to the development of allele specific therapies.

According to another embodiment of the invention, the nucleic acid sample may comprise at least one allele. Preferably, the UV absorption of a candidate allele is compared to the UV absorption of a wild type allele with and without the bound probe at a variety of temperatures. In this embodiment, the method may be carried out by using a plurality of channels so that the nucleic acid sample comprising at least one candidate allele is passed through one channel and the nucleic acid sample comprising the wild type allele is passed through a second channel.

In a further aspect, the invention relates to the use of an apparatus in the method of any of the preceding claims having a plurality of temperature-controlled channels.

The main advantages of the proposed system are, but not limited to:

Use of a non-PCR based system for SNP validation is highly desirable. SNP validation is highly dependent on the systems ability to detect single base changes out of many hundred on the target template. A crucial problem with PCR is that there are error rates inherent in all thermostable and other DNA polymerases—this is a crucial function in evolutionary processes. Table below shows common error rate details of common polymerases (Michael Borns et al, Comparing PCR Performance and Fidelity of Commercial Pfu DNA polymerases, http://www.biocompare.com/techart.asp?id=121).

TABLE 1

| | Current Assay Results | | Published lacI Assay Results | |
|---|---|---|---|---|
| | | Mean Error | | Mean Error Rate |
| | Number of | Rate (×10⁻⁶) | Number of | (×10⁻⁶) |
| Polymerase | PCRs | (±range) | PCRs | (±range) |
| Cloned Pfu | 2 | 1.3 ± 0.1 | 10 | 1.3 ± 0.2¹ |
| pfuturbo ® | 2 | 1.3 ± 0 | — | — |
| Native Pfu | 2 | 2.0 ± 0 | 4 | 0.8 ± 0.2⁹* |
| (Stratagene) | | | | |
| Native Pfu | 2 | 2.0 ± 0.5 | — | — |
| (competitor) | | | | |
| Deep Vent ™ | — | — | 4 | 2.7 ± 0.2¹ |
| Vent ® | — | — | 6 | 2.8 ± 0.9¹ |
| Taq | — | — | 11 | 8.0 ± 3.9¹ |
| | | | 4 | 10.0 ± 0.2⁹* |

*Error rates were recalculated from reference 9 to reflect an updated lacI target size of 349 bp¹

The removal of this potential area of error would be extremely beneficial in this process. PCR also carries inherent cost implications as the components of the reaction can form a major budget item in any SNP program of work. The TaqMan system is even more expensive. There are 100s of thousands of potential SNPs (International SNP Map Working Group 2001. Nature 409, pp 928-934: A map of human genome sequence variation containing 1.42 million single nucleotide polymorphisms) on the human genome, suggesting an immense capital outlay in order to investigate them in an efficient manner. If this is coupled to the need for ‘personalised’ medicine, where individuals will benefit from SNP analysis prior to drug therapy, only a non-PCR system will be feasible. The proposed system carries neither of these penalties.

In the methods of the invention, UV absorption may be measured using UV sensitised Photo Diode Arrays (PDAs) or charge-coupled devices (CCDs). For example, as shown in FIG. 1, the light source (such as a Deuterium lamp or UV diode laser), optical parts (UV lenses), separation phase and detector are arrayed on a common rail. Light from the low-noise deuterium lamp or UV diode laser passes through a filter wheel allowing the selection of detection wavelength. The light is then focused on a fused silica capillary, typically with an internal diameter of 50-100 micrometers (μm). As the nucleic acid passes the light beam, it absorbs energy dependent on its spectral characteristics. The light beam is then focused on to the detector where the drop in signal due to the energy absorbed by the nucleic acid is measured.

### DESCRIPTION OF THE FIGURES

The invention will be further understood by reference to the non-limiting drawings.

FIG. 1 shows the optical layout.

FIG. 2 shows the temperature control system. Peltier cells, Air or Liquid heating, Joule heating or any other applicable system could control the temperature environment.

FIG. 3 shows a microfluidic chip or capillary.

FIG. 4 illustrates non-PCR based SNP analysis. Due to the sensitivity of the system used, the invention aims at detecting the melting temperature of primer/template complexes. Therefore using only one allele specific primer or probe to probe two possible alleles it may be possible to measure the affinity of the allele specific primer to the template by melting temperature analysis. If a SNP is present the probe/template complex will be less stable and exhibit a lower melting temperature. Even if the difference is very small, the temperature resolution of the method is 0.1° C. which should allow this analysis and the increase due to the hyperchromic effect will be measurable.

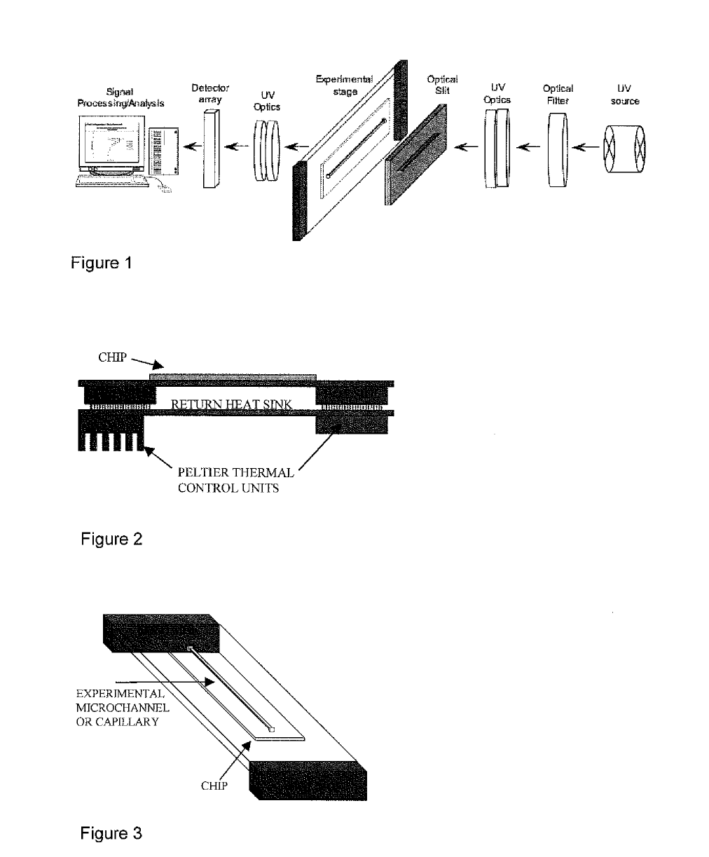

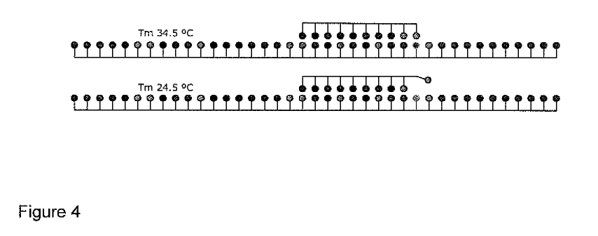

1. A method for the detection of a nucleotide modification in a nucleic acid sample comprising:
a) contacting a solution comprising a nucleic acid sample with a nucleic acid probe in a temperature-controlled and UV illuminated container;
b) measuring the UV absorption of the nucleic acid sample/nucleic acid probe complex;
wherein the method does not comprise nucleic acid amplification.

2. The method of claim 1 wherein the method comprises analysing the Tm of the complex.

3. The method of claim 1 or claim 2 wherein the container is a temperature-controlled and UV illuminated channel.

4. The method of claim 3 wherein the nucleic acid sample/nucleic acid probe complex is passed along the channel.

5. The method of claim 3 or 4 wherein the nucleic acid sample/nucleic acid probe complex is imaged along the channel.

6. The method of any preceding claim wherein the nucleic acid sample comprises DNA or RNA.

7. The method of any preceding claim wherein the nucleic acid is single stranded or double stranded.

8. The method of any preceding claim wherein UV absorption is measured at a range of temperatures.

9. The method of claim 8 wherein the range of temperature is from about 20° C. to about 110° C.

10. The method of any preceding claim wherein the UV absorption is measured as a function of the temperature.

11. The method of any preceding claim wherein the presence or absence of double stranded nucleic acid is detected.

12. The method of any preceding claim wherein the nucleic acid sample comprises at least one allele.

13. The method of any preceding claim wherein the UV absorption of a candidate allele is compared to the UV absorption of a wild type allele.

14. The method of any preceding claim wherein the nucleotide modification is SNP.

15. The method of claim 14 wherein the SNP is associated with disease.

16. The method of any preceding claim wherein the nucleotide modification is detected in real time.

17. The method according to any preceding claim wherein the nucleic acid probe is labelled.

18. The method according to any of claims 1 to 16 wherein the nucleic acid probe is not labelled.

19. The use of the method of any preceding claim for diagnosing a patient as having a disease or being susceptible to it.

20. The use of an apparatus in the method of any preceding claim having a plurality of temperature-controlled channels.
